(19) United States
(12) Patent Application Publication
(10) Pub. No.: US 2021/0030581 A1
(43) Pub. Date: Feb. 4, 2021

(54) SENSOR FOR LOCATING WITHIN A URINARY DRAINAGE BAG

(71) Applicant: THE UNIVERSITY OF BATH, Bath Bath and North East Somerset (GB)

(72) Inventors: Scarlet MILO, Bath and North East Somerset (GB); Andrew Tobias JENKINS, Bath and North East Somerset (GB)

(21) Appl. No.: 16/964,366

(22) PCT Filed: Jan. 28, 2019
(86) PCT No.: PCT/GB2019/050225

§ 371 Date: Jul. 23, 2020

(30) Foreign Application Priority Data
Jan. 29, 2018 (GB) 1801403.5

(51) Int. Cl. A61F 5/44; A61M 25/00; A61L 31/14; A61L 31/06; C12Q 1/04

(52) U.S. Cl. CPC A61F 5/44; A61M 25/0017; A61L 31/145; A61M 2205/3324; C12Q 1/04; A61M 2205/584; A61L 31/06

(57) ABSTRACT

The present invention provides a sensor for locating within a urinary drainage bag, the sensor comprising an agent held by a pH-sensitive release material; wherein the agent is a diagnostic marker and wherein the pH-sensitive release material is configured to release the agent when the pH-sensitive release material is exposed a pH above 7, together with a urinary drainage bag comprising the same. Methods for preventing catheter blockage using the sensor and methods for manufacturing the sensor are also provided.

### FIELD OF INVENTION

The present invention provides a sensor for locating within a urinary drainage bag, methods for preventing urinary catheter blockage and methods for manufacturing sensors.

### BACKGROUND TO THE INVENTION

Catheter-associated urinary tract infection (CAUTI) accounts for approximately 80% of nosocomial infections worldwide. Foley catheters are often used on a long-term (30 days) indwelling basis as a common management technique for urinary incontinence or retention, and are universally complicated by polymicrobial and dynamic bacteriuria. Specifically, infections caused by the Gram-negative, motile bacterium Proteus mirabilis (P. mirabilis) comprise 20-45% of catheter-related infections, since P. mirabilis readily colonises all available catheter types to form extensive biofilm communities. Expression of a potent urease enzyme catalyses the hydrolysis of urea in the urine, resulting in a rapid pH increase. The induced alkaline environment initiates precipitation of polyvalent ions (including struvite (MgNH₄PO₄·6H₂O) and carbonate-apatite [Ca₁₀-(PO₄CO₃OH)₆(OH)₂]). Local supersaturation and subsequent accumulation of crystalline biofilms continues within the catheter until the flow of urine is obstructed, resulting in painful distention of the bladder, and serious symptomatic episodes such as acute pyelonephritis and septicaemia.

Since many long-term catheterised patients are in community care, bacteriological analysis of their urine is rarely performed. Consequently, colonisation by P. mirabilis often remains undetected until the emergence of severe sequelae. Indeed, there is currently no reliable way of preventing or accurately predicting when blockage may occur. Moreover, patients who commonly suffer from catheter blockage may have regular scheduled catheter changes in their care plan; however the irregularity of encrustation and blockage often results in needless or emergency changes, instigating patient trauma and avoidable healthcare costs.

The concept of using urinary pH elevation to provide an ‘early warning’ of catheter blockage has been explored previously via the covalent linkage of the pH indicator bromothymol blue to cellulose acetate polymer. Despite successful in vitro (Stickler et al., 2006a) and clinical assessments (Stickler et al., 2006b) of this system, this sensor was deemed unsuitable for commercial development. Hence, the system was modified to improve commercial viability via the manufacture of a silicone-based formulation with the incorporated indicator and hydrophilic filler. The sensor was housed within a small polyvinyl chloride connecter, located in the junction between catheter and drainage bag, and initially proved promising both in vitro and in clinical studies. However, the ‘early warning’ of catheter blockage in human trials was >18 days, thus calling into question whether this system can be considered an indicator of imminent catheter blockage.

Consequently, there is a need for devices to provide early diagnosis of a bacterial infection, especially an infection caused by bacterial strains capable of inducing an elevated pH in a patient's urine.

### SUMMARY OF THE INVENTION

In a first aspect the present invention provides a sensor for locating within a urinary drainage bag, the sensor comprising an agent held by a pH-sensitive release material; wherein the agent is a diagnostic marker and wherein the pH-sensitive release material is configured to release the agent when the pH-sensitive release material is exposed a pH above 7.

Sensors of the present invention can be placed directly into a catheter urine bag and typically have the form of a small lozenge or capsule. The sensor is able to signal impending catheter encrustation and blockage following infection by bacteria such as P. mirabilis. Elevation of urinary pH following P. mirabilis infection causes the pH sensitive release material to release the diagnostic marker, typically resulting in a clear visual colour change within the catheter drainage bag. Unlike previously reported technology, the sensor of the present invention avoids potential adverse in vivo effects such as urethral inflammation, or promotion of microbial adhesion via deposition of a conditioning film on the luminal and external catheter surfaces. Furthermore, localised diagnostic marker release within the drainage bag, without the spatial constraints of the catheter tip, allows for a greater concentration of diagnostic marker to be released within the visible portion of the catheterised urinary tract, which can result in a clear visual colour change of residual urine to warn of impending blockage and allow sufficient time for clinical intervention.

The present invention also provides a urinary drainage bag comprising a sensor of the invention, and a urinary catheter comprising such a drainage bag.

In a further aspect the present invention provides a method of preventing urinary catheter blockage due to bacterial colonisation, the method comprising the steps of: fitting a urinary catheter comprising a drainage bag containing a sensor of the present invention; and monitoring release of the diagnostic marker, wherein release of the diagnostic marker indicates bacterial colonisation.

The present invention also provides a method for manufacturing a sensor, the method comprising: combining a solution of a diagnostic marker and a hydrogel polymer and heating the solution to form a hydrogel containing the diagnostic marker, cooling the hydrogel containing the diagnostic marker to about −20° C. to promote cryogenic gelation, thawing the hydrogel containing the diagnostic marker at room temperature, and coating the thawed hydrogel containing the diagnostic marker with a pH-sensitive release material.

### DESCRIPTION

According to a first aspect the present invention provides a sensor for locating within a urinary drainage bag, the sensor comprising an agent held by a pH-sensitive release material; wherein the agent is a diagnostic marker and wherein the pH-sensitive release material is configured to release the agent when the pH-sensitive release material is exposed a pH above 7.

The sensor is typically a unit dose article and may be provided in the form of a lozenge or tablet that can easily be placed within a urinary drainage bag. The shape of the sensor is not particularly limited and may be spherical, cylindrical, or any convex polyhedron. Other sensor shapes include those commonly used for tablets, which will be familiar to the skilled person, and include round, oval, almond, square, rectangular and pillow shaped.

The pH-sensitive release material is configured to release the diagnostic marker when the pH-sensitive release material is exposed to a pH above 7. That is, the diagnostic marker is released when the pH-sensitive release material is exposed to a pH between 7 and 14. Specifically, the pH at which the pH-sensitive release material releases the diagnostic marker is between 7 and 14. For example, the sensor may include a pH-sensitive release material that releases the diagnostic marker at or above pH 7 but not below pH 7.

The pH-sensitive release material can release the diagnostic marker when the pH-sensitive release material is exposed to a pH above 7, for example, above 7.1, 7.2, 7.3, 7.4, 7.5, 7.6, 7.7, 7.8, 7.9, 8.0, 8.2, 8.4, 8.6, 8.8, 9.0, 9.5 or above 10.0. In preferred embodiments of the invention the pH-sensitive release material is exposed to a fluid having an elevated pH in order to trigger release of the diagnostic marker. An elevated pH may refer to a pH of 7 or more.

By locating the sensor within a urinary drainage bag, the sensor is in direct contact with a patient's urine. This has the advantage of ensuring that the pH-sensitive release material is exposed to the urine to allow for release of the diagnostic marker when the urine pH reaches the required value. The diagnostic marker is preferably held, or trapped, within the sensor until the pH-sensitive release material is exposed to a pH of 7 or more.

In preferred embodiments of the invention, the sensor comprises an inner layer containing the diagnostic marker and an outer layer comprising the pH-sensitive release material. The inner layer therefore acts as a reservoir for the diagnostic marker. Without being bound by theory, it is believed that the outer layer (comprising the pH-sensitive release material) acts to prevent the diagnostic marker leaving the inner layer. In the presence of an elevated pH the outer layer is broken down, releasing the diagnostic marker into the urinary drainage bag.

By separating the diagnostic marker and the pH-sensitive release material into two or more layers, manufacture of the sensor is versatile and simple. For example, formulations of particular layers can be changed independently of each other and thicknesses of each layer can be built up independently of each other. Preferably, the inner layer (containing the diagnostic marker) is covered by the outer layer (comprising the pH-sensitive release material), which protects the inner layer from the environment. In preferred embodiments of the invention the inner layer of the sensor is completely covered by the outer layer comprising the pH-sensitive release material. In other words, in preferred embodiments of the invention 100% of the inner layer is covered by the outer layer.

The inner layer of the sensor (containing the diagnostic marker) can comprise a hydrogel or a liquid. Hydrogels are particularly suitable carriers for the diagnostic marker as hydrogels have the ability to allow the diagnostic marker to diffuse out. Hydrogel as used herein refers to a system in which hydrophilic polymer chains are dispersed in an aqueous solution, such as an aqueous buffer solution or water. Preferably the hydrogel comprises at least 70% w/v aqueous solution. In embodiments of the invention the hydrogel comprises about 80% w/v aqueous buffer solution. Typically, the hydrogel is in a gel state, such as a semi-solid state which retains shape. Suitable hydrogels may be naturally occurring or synthetic. Naturally occurring hydrogels include agarose, gelatine, gellan gum, carrageenan, pectin, xanthan gum, agar, chitosan, phytagel and methylcellulose as well as synthetic derivatives of naturally occurring hydrogels, such as poly-captolactone and poly-hydroxyethyl methacrylate. In preferred embodiments of the invention the hydrogel includes poly(vinyl alcohol), agarose, hypromellose (i.e. hydroxypropyl methylcellulose (HPMC)), gelatine, sodium polyacrylate or mixtures thereof. In particularly preferred embodiments of the invention the hydrogel comprises (poly (vinyl-alcohol) (PVA)).

In embodiments of the invention the inner layer of the sensor comprises the diagnostic marker and a hydrogel at a ratio about 1:1.

The pH-sensitive release material can comprise any material that is initially impermeable to the diagnostic marker. By impermeable, we mean that the quantity of diagnostic marker that can break through the pH-sensitive release material is negligible in terms of diagnostic quantities. The material should maintain impermeability while exposed to pH values below 7 but become permeable to the diagnostic marker when the material is exposed to urine with a pH of 7 or more. At this point, diagnostic quantities of the diagnostic marker can permeate the pH-sensitive release material.

If desired, the pH-sensitive release material may be an intelligent material that can reversibly switch between permeability and impermeability. Typically, however, permeability is afforded more simply and cost-effectively by a pH-sensitive material that degrades in response to a fluid of appropriate pH. Such materials are well known in the art and include, for example, materials used for enteric coatings, which will be familiar to the skilled person. Such pH-sensitive materials present a stable surface at an acidic pH (i.e. below 7) but will break down when exposed to a less acidic pH (i.e. above 7), thereby releasing the agent. Suitable pH sensitive materials include pH-sensitive polymers comprising carboxyl and ester groups. pH-sensitive polymers may comprise carboxyl and ester groups at a ratio of 1:2. In preferred embodiments of the invention the pH-sensitive release material is a pH-sensitive polymer comprising poly(methyl methacrylate-co-methacrylic acid).

In embodiments of the invention the inner layer of the sensor (containing the diagnostic marker) can be encapsulated, e.g. in a gelatine or hydroxypropyl methylcellulose (HPMC) capsule. The capsule can then be coated with the pH-sensitive release material.

The diagnostic marker can comprise a dye. This means that a detectable signal is released when the required pH change occurs. The type of dye is not particularly limited. However, it is preferred that release of the dye can be detected by the naked eye. Therefore, it is preferred that the dye has a strong and obvious colour, which is visible under normal visible light. In other words, it is preferred that the dye has an emission wavelength in the normal visible light range. This helps to ensure quick and simple detection without the need for complex equipment or specialist training. Preferably, the dye can be detected at low concentrations. The dye is preferably biocompatible and non-irritant to the patient. This avoids further aggravation of the patient, and is further assisted by a dye that can be detected at low concentrations. Fluorophoric dyes are particularly suitable for detection at low concentrations and suitable for use in the present invention. The dye may be a self-quenching fluorescent dye. Fluorophoric dyes that can be used in invention include fluorescein, carboxyfluorescein, sulforhodamine B or combinations thereof. In preferred embodiments of the invention the dye is carboxyfluorescein.

In embodiments of the invention the inner layer of the sensor comprises a diagnostic marker, such as a dye, at a concentration of about 10 mM to about 100 mM, preferably about 20 mM to about 70 mM, more preferably about 40 mM to about 60 mM. In embodiments of the invention the inner layer comprises the diagnostic marker at a concentration of about 50 mM. Alternatively, the inner layer may comprise the diagnostic marker at a concentration of about 100 mM to about 500 mM, preferably about 200 mM to about 300 mM. In preferred embodiments of the invention the inner layer may comprise the diagnostic marker at a concentration of about 250 mM.

In embodiments of the invention the sensor comprises a cylindrical hydrogel reservoir, containing a self-quenching fluorescent dye. The hydrogel matrix is completely encapsulated and sealed by a pH-sensitive release layer. Elevation of urinary pH following P. mirabilis infection causes swelling of the release layer, releasing the dye to result in a clear visual colour change within the catheter drainage bag. In embodiments of the invention the hydrogel is poly (vinyl-alcohol) (PVA). The self-quenching fluorescent dye may be 5(6)-carboxyfluorescein (CF). The pH-sensitive release layer may be composed of EUDRAGIT® S100 (poly(methyl methacrylate-co-methacrylic acid)).

In alternative embodiments of the invention the sensor comprises a gelatine or HMPC capsule, containing a self-quenching fluorescent dye. The capsule is completely coated by a pH-sensitive release layer. Elevation of urinary pH following P. mirabilis infection causes swelling of the release layer, releasing the dye from the capsule to result in a clear visual colour change within the catheter drainage bag. The self-quenching fluorescent dye may be 5(6)-carboxyfluorescein (CF). The pH-sensitive release layer may be composed of EUDRAGIT® S100 (poly(methyl methacrylate-co-methacrylic acid)). The capsule may contain the self-quenching fluorescent dye at a concentration of about 250 mM. Preferably the dye is encapsulated in its liquid form, meaning that no hydrogel is added to the capsule.

The sensor may comprise multiple diagnostic markers held by different pH-sensitive materials that release different diagnostic markers at different pH values. For example, dyes of different colours may be held such that one colour is released at a first pH (when the infection is not yet well established) and another colour is released at a second pH (when the infection is well established). This can be achieved, for example, by separating the diagnostic markers/pH-sensitive materials into different layers of the sensor.

The present invention additionally provides a urinary drainage bag comprising a sensor as described herein. Also provided is a urinary catheter comprising such a drainage bag. Typically, the catheter is an indwelling catheter. When in use a portion of the catheter dwells within the bladder within the patient's body and the urinary drainage bag will be outside the patient's body. Specifically, a portion of the catheter, usually the tip of the catheter with the drainage eyelet, dwells within the bladder.

Urine flows from the bladder, through the catheter and into the drainage bag containing the sensor. If a microbial infection raises the pH of the urine the diagnostic marker, which may be a dye, is released into the drainage bag, signalling the presence of an infection. This can allow an earlier diagnosis than would otherwise have been possible and is particularly beneficial when treating or preventing catheter associated urinary tract infections, which can lead to encrustation and blockage of the catheter, with possible kidney damage if not detected or treated in time.

The present invention therefore additionally provides a method of preventing urinary catheter blockage due to bacterial colonisation, the method comprising the steps of: fitting a urinary catheter comprising a drainage bag containing a sensor as described herein to a patient, and monitoring release of the diagnostic marker, wherein release of the diagnostic marker indicates bacterial colonisation.

The diagnostic marker is preferably released from about 12 to about 18 hours prior to catheter blockage. More preferably the diagnostic marker is release about 14 hours prior to catheter blockage.

In embodiments of the invention the diagnostic marker may be released within about 8 hours of a bacterial infection being developed. Preferably the diagnostic marker is released within about 6 hours or within about 4 hours of a bacterial infection being developed. In preferred embodiments of the invention substantially all of the diagnostic marker is released within about 18 hours of a bacterial infection developing, more preferably substantially all of the diagnostic marker is released within about 14 hours of a bacterial infection developing.

The bacterial strain can be urease producing. This is particularly relevant to urinary tract infections, where activity of the urease enzyme generates ammonia and raises pH. The bacterial strain can be one or more of Proteus mirabilis, Providencia rettgeri, Morganella morganii, Pseudomonas aeruginosa and Proteus vulgaris or combinations thereof. In urinary tract infections, these bacterial strains are known to elevate the pH of urine.

The method may further comprise removing the catheter from the patient after release of the diagnostic marker. Preferably the catheter is removed from the patient within about 18 hours of release of the diagnostic marker.

The patient is preferably a mammal, more preferably a primate, especially a human, and may be a paediatric or geriatric patient.

The present invention additionally provides a method for manufacturing a sensor as described herein, the method comprising: combining a solution of a diagnostic marker and a hydrogel polymer and heating the solution to form a hydrogel containing the diagnostic marker, cooling the hydrogel containing the diagnostic marker to about −20° C. to promote cryogenic gelation, thawing the hydrogel containing the diagnostic marker at room temperature, and coating the thawed hydrogel containing the diagnostic marker with a pH-sensitive release material.

The diagnostic marker and hydrogel polymer are preferably combined at a ratio of about 1:1. For example, a solution of diagnostic marker, such as a dye, may be prepared at a concentration of about 50 mM and adjusted to about pH 6 prior to adding the hydrogel (optionally at a concentration of about 10% w/v). The solution may then be heated to about 97° C., preferably with constant stirring to facilitate diffusion. In embodiments of the invention the thawed hydrogel containing the diagnostic marker may be coated by dip coating. Dip coating may be repeated as necessary, for example 10 to 90 times, or 30 to 70 times or 50 times, optionally with a solvent evaporation period at room temperature between each coating. Alternatively, the thawed hydrogel containing the diagnostic marker may be capsulated in a capsule, such as a commercially available gelatine or HMPC capsule, which may be subsequently coated with the pH-sensitive release material. The capsule may be coated by e.g. spray coating or tablet coating. Coated sensors may be stored at 4° C. until required.

In an alternative embodiment of the invention the sensor many be manufactured by a method comprising filling a capsule with a diagnostic marker in liquid form, sealing the capsule and then coating the sealed with capsule with a pH-sensitive release material, such as Eudragit S100. Suitable capsule coating processes will be familiar to the skilled person and may include, e.g. spray coating or tablet coating. The capsule may be a gelatine capsule or an HPMC capsule. The diagnostic marker may be at a concentration of about 250 mM.

### BRIEF DESCRIPTION OF THE DRAWINGS

The invention will now be described in detail, by way of example only, with reference to the figures.

FIG. 1 shows dimensions of the pH-responsive polymeric lozenge sensors (dual-layered lozenge sensors containing an inner core of self-quenched 5(6)-carboxyfluorescein (25 mM) and an outer shell of the pH-responsive polymer EUDRAGIT® S100.), showing the positioning of the sterile thread.

FIG. 2 shows encapsulation efficiency of 30 coats of Eudragit S100 at pH 6 and 8. Leaching of the fluorescent dye was observed upon room-temperature incubation of the lozenge sensor within phosphate buffer simulating healthy urine pH.

FIG. 3 shows encapsulation efficiency of 40 coats of Eudragit S100. Visual dye release was observed after 23 hours incubation in phosphate buffer at pH 6 and 8.

FIG. 4 shows encapsulation efficiency of 50 coats of Eudragit S100 at pH 6 and 8. Coatings remained stable for the duration of the 24 hours incubation at healthy urine pH. Rate of dye release into the recipient medium.

FIG. 5 shows analysis of 5(6)-carboxyfluorescein properties. A) Dependence of fluorescence emission on CF concentration at pH 6, 7 and 8, showing self-quenching at 10 mM. (Inset: visual representation of CF fluorescence at concentrations 50 nM-50 mM at pH 8). B) Dependence of fluorescence emission on pH. (Inset: chemical structure of CF). Data shown is the mean of triplicate repeats. Error bars represent standard error of the mean (SEM).

FIG. 6 shows analysis of sensor performance in 1:1000 artificial urine subcultures, modelling the onset of catheter-associated urinary tract infection. A) pH elevation of artificial urine subcultures of P. mirabilis B4 and E. coli NSM59. B) Corresponding fluorescence emission of prototype sensors upon release of CF dye into artificial urine subcultures. Data shown is the mean of triplicate repeats. Error bars represent SEM.

FIG. 7 shows determination of 5(6)-carboxyfluorescien release from pH-sensitive lozenge sensors at pH 7 and 8. Empirical dye release was monitored over the course of 120 minutes at physiological pHs (pH 6, 7 and 8) (A). Initial release was defined as the first 15 minutes of release, within which the fluorescence output was linear (B). Standard curves of fluorescence intensity as a function of [carboxyfluorescein] at pH 7 (C) and pH 8 (D) were used to evaluate the kinetic release of dye from the sensors at each pH (E, F for pH 7 and 8 respectively). Rate of release could not be analysed at pH 6, owing to negligible dye release. Data shown are the mean of triplicate repeats. Error bars represent SEM.

FIG. 8 shows analysis of sensor performance in artificial urine supernatants of P. mirabilis and E. coli species. Average pH of supernatants was 8.98 for urease-positive and 6.10 for urease-negative species, thus mimicking the urine of infected and healthy patients, respectively.

FIG. 9 shows evaluation of the point of sensor ‘switch on’, using 1:1000 subculture of P. mirabilis in artificial urine. Urinary pH elevation occurs as a result of urease-mediated urea hydrolysis. Corresponding increase in bacterial bioburden is observed during the transition from lag phase to exponential growth of P. mirabilis.

FIG. 10 shows: A) Quantitative analysis of dye release into residual artificial urine at experiment start, point of visual colour change and catheter blockage. B) Appearance of drainage bag at experiment start (0 hours). C) Point of qualitative visual colour change (3 hours) in bladder model infected with 10⁸ CFU/ml P. mirabilis B4. D) Colour change of artificial urine at end of experiment (17.5 hours) in bladder model inoculated with P. mirabilis B4. E) Appearance of catheter bag at end of experiment (17.5 hours) in bladder model inoculated with 10⁸ CFU/ml E. coli NSM59. No visual colour change was observed for the urease-negative species. Data shown is the mean of triplicate repeats. Error bars represent SEM.

### EXAMPLE 1

### Methodology

### Microbiological Methods

### Preparation of Bacterial Supernatants

Bacterial overnight cultures (grown from a single bacterial colony), were centrifuged (4,000 rpm, 20 minutes) to pellet whole cells. The liquid phase of each sample was collected, sterilised by filtration (0.22 μm pore size) to remove any residual cells, and used immediately. Sedimented cells were discarded.

### Preparation of Bacterial Subcultures

Bacterial overnight culture (10 μL in LB media) was added to artificial urine media (10 mL). 1:1000 subcultures were grown at 37° C. with agitation to assess sensor response.

### Correlation of Viable Cell Count with Urinary pH

The point at which sensor ‘switch on’ was achieved was evaluated using 1:1000 subcultures of P. mirabilis B4 and E. coli NSM59. Cultures were grown into the exponential phase over 3 hours. Samples (100 μL) were removed at 10 minute intervals for analysis of bacterial bioburden (via serial dilution and plating on NSLB agar). Plates were incubated overnight and viable cell numbers quantified via colony counting. Measurements of subculture pH and fluorescence were also recorded at 10 minute intervals using an electronic pH meter (Jenway 3540), and microplate reader, respectively.

### Analysis of 5(6)-Carboxyfluorescein Properties

### pH-Dependency

The pH-dependent response of CF was assessed via pH adjustment of 0.5 mM CF solution (in HEPES buffer) within the range of pH 2-10 in 0.5 increments. A SPECTROstar Omega microplate reader (BMG Labtech, UK) was used to monitor fluorescence endpoints, using excitation and emission wavelengths of 485±12 and 520 nm, respectively.

### Concentration Quenching

Dilutions from CF stock (50 mM) were undertaken to achieve a concentration range of 10 mM-10 nM in HEPES buffer. Dilutions were adjusted to pH 6, 7 and 8 accordingly via dropwise addition of NaOH/HCl (1M). Fluorescence endpoint measurements were read on a microplate reader.

### Preparation of EUDRAGIT® S100 Solution

An organic dip coating solution of EUDRAGIT® S100 (average molecular weight of 150,000 g/mol) (Evonik industries, Germany) was prepared as previously reported (Milo et al., 2016) and stored at room temperature until required.

### Dip Coating Optimisation

Lozenge sensors were prepared according to “Preparation of pH-Sensitive Lozenge Sensors (2)” (below), and dip coated with 30, 40 or 50 coats of Eudragit S100 solution to assess dye retention with varying coating thickness. Release was assessed via overnight incubation at room temperature in phosphate buffer (10 mL) at pH 6 and 8. Fluorescence output was monitored using a microplate reader.

### Preparation of pH-Sensitive Lozenge Sensors (1)

To CF solution (50 mM, adjusted to pH 6) was added PVA (14,600-18,600 g/mol) (10% w/v) and heated to 97° C. with constant stirring to facilitate diffusion. The resultant hydrogel (1 ml) was cast into a 24-well microplate containing a 2 cm length of sterilised cotton thread, and stored at −20° C. overnight to promote cryogenic gelation. Sensors were thawed at room temperature (4 h) before dip coating with the EUDRAGIT® S100 trigger layer via suspension from the thread. Sensors were manually dip-coated 50 times, with a 5 minute solvent evaporation period at room temperature between each coating. Coated sensors were stored at 4° C. until required.

### Preparation of pH-Sensitive Lozenge Sensors (2)

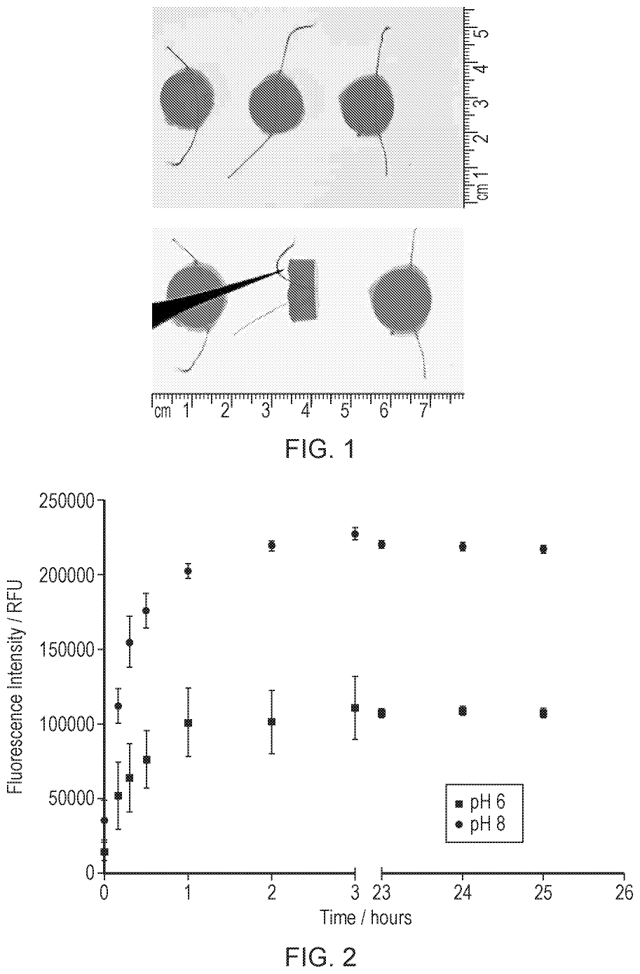

To carboxyfluorescein solution (250 mM, adjusted to pH 6) was added PVA (14,000-18,000 g/mol, 15% w/v) and heated to 97° C. with constant stirring to facilitate diffusion. The resultant hydrogel (1 mL) was cast into a 24-well microplate containing a 2 cm length of sterilised cotton thread (FIG. 1), and stored at −20° C. overnight to promote cryogenic crosslinking.

Sensors were thawed at room temperature (4 hours) before dip coating with Eudragit S100 trigger layer via suspension from the thread. Sensors were manually dip-coated 50 times, with a 5 minute solvent evaporation period at room temperature between each coating. Coated sensors were stored at 4° C. until required.

### Evaluation of Sensor Performance

### In Vitro Bladder Models

Bladder model setup and operation was followed as previously reported (Milo et al., 2016). Artificial urine was prepared according to (Stickler et al., 1999) and supplied to the bladders via a peristaltic pump at a flow rate of 0.75 ml/min. Models simulating late-stage infection were inoculated directly with clinical isolates of P. mirabilis B4 or E. coli NMS59 bacteria (10⁸ colony forming units/ml (CFU/ml)). Two individual sensors were added aseptically to each drainage bag, and observed visual changes in urine colour were correlated with measured fluorescence response within the drainage bag. Fluorescence emission was quantified using a microplate reader as previously described.

### Bacterial Cultures

Prototype sensors were evaluated for species selectivity using live cultures of P. mirabilis B4 and E. coli NSM59 clinical isolates (˜5×10⁶ CFU/ml). To artificial urine media (10 ml) was added bacterial overnight culture (10 μl). Coated sensors were added to subcultures, and changes in pH, and fluorescence response as a function of dye release were measured via sequential sampling.

### Kinetics of Carboxyfluorescein Release

To evaluate the quantity of, and rate at which the carboxyfluorescein dye is released from the sensors, the kinetics of dye release was analysed at pH 6, 7 and 8 (37° C., phosphate buffer). Dye release from the lozenge sensors was monitored at regular time intervals, and subsequent graphs of initial release (first 15 minutes) were created. Standard curves of fluorescence intensity as a function of carboxyfluorescein concentration were also produced at pH 6, 7 and 8 (linear portion only). Values of dye concentration were calculated from the standard curves in order to achieve graphs of carboxyfluorescein concentration vs. time from the original sensor release. Kinetic evaluation was not completed for pH 6, as release of dye was negligible. The gradient of the resultant graphs at pH 7 and 8 shows the rate (mol dm⁻³ min⁻¹) of carboxyfluorescein release within the first 15 minutes of contact.

### Results and Discussion

### Eudragit S100 Dip Coating Optimisation

Since the lozenge sensor design incorporates a significantly larger volume of PVA reservoir than the original catheter coating, it was predicted that a considerably thicker film of the Eudragit S100 trigger layer would be required to provide sufficient entrapment efficiency of the dye within the hydrogel. Encapsulation of 5(6)-carboxyfluorescein was investigated in solutions of phosphate buffer at pH 6 and 8 to simulate healthy and infected urine, respectively. Film thickness (30, 40 or 50 dip coats) was deemed sufficient when a visible fluorescence output was obtained at pH 8, but not at pH 6.

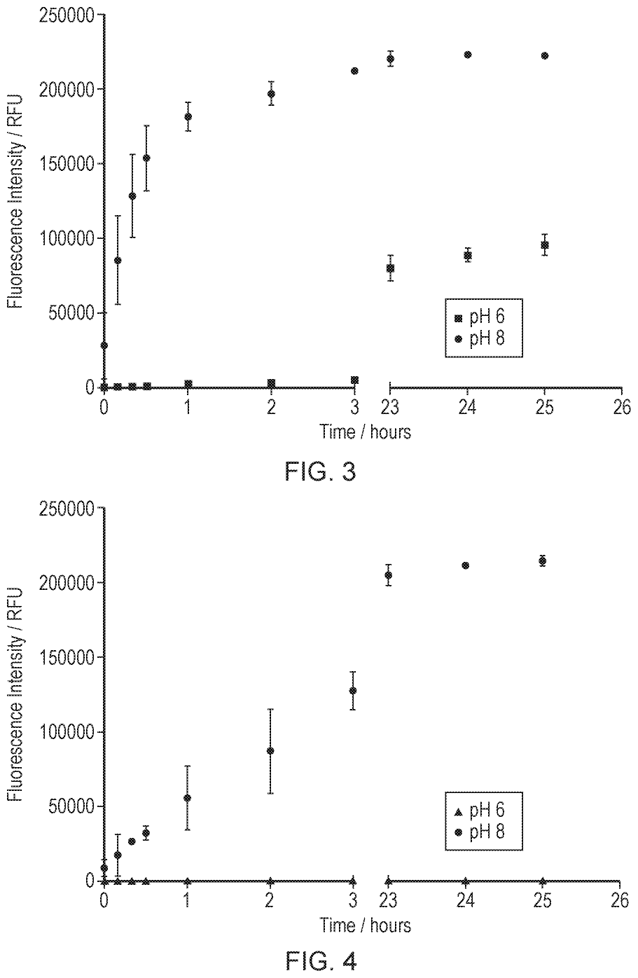

Dye retention within the hydrogel reservoir was insufficient after 30 coats of the polymeric trigger (FIG. 2). Significant dye release was observed within the first hour of incubation in buffer solution mimicking healthy urine pH. When the film thickness was increased to 40 coats, carboxyfluorescein dye was more successfully retained within the central hydrogel region of the lozenge sensor (FIG. 3).

Lozenge sensors appeared stable at pH 6 after 3 hours incubation, as dye release and subsequent fluorescence ‘switch on’ was insufficient to cause a visual response. However, after overnight incubation, the structural integrity of the Eudragit shell was compromised, resulting in leaching of the loaded dye into the bulk solution. Dye release at pH 6 was less efficient than pH 8, yet sufficient to cause a visual colour change within the recipient medium.

Analysis of dye retention within lozenge sensors coated with 50 dip coats of Eudragit S100 showed effective encapsulation for the duration of the assay (FIG. 4). Loaded dye was effectively maintained within the hydrogel reservoir in the presence of buffer mimicking healthy urine pH, but underwent a rapid and visually unambiguous fluorescence ‘switch on’ at pH representing urine infected with urease-positive species. The rate of dye release appeared slower with increasing coating thickness, although still resulted in visual fluorescence within the first hour of incubation.

Overall, analysis of carboxyfluorescein retention showed that dye encapsulation within the hydrogel matrix is dependent on Eudragit film thickness. Prototype production will henceforth be completed with 50 coats of Eudragit polymer, as this provides the optimum compromise between sensor stability and production time.

### Analysis of 5(6)-Carboxyfluorescein

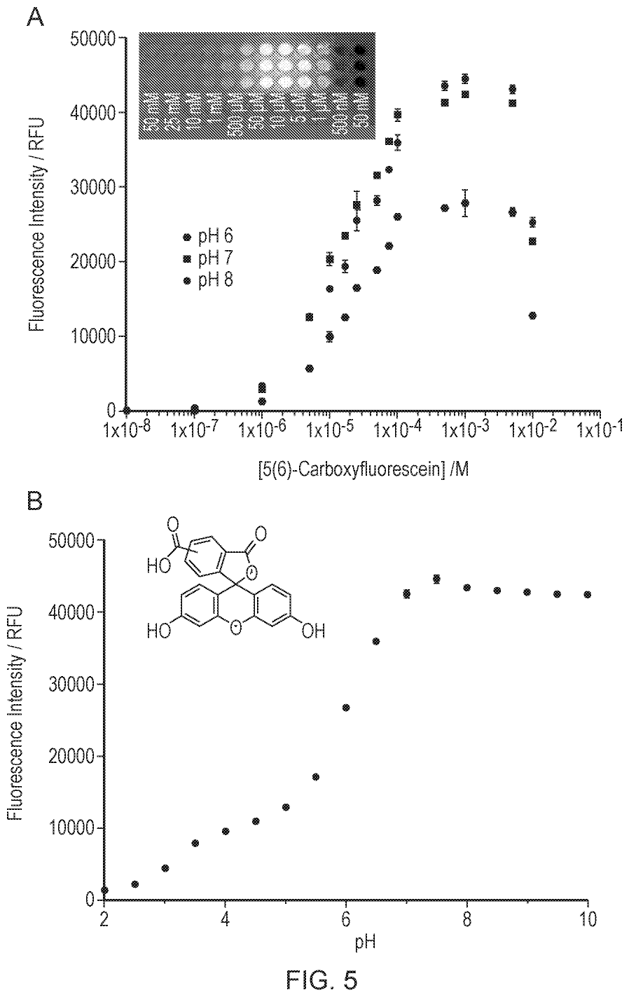

The suitability of CF for the in-situ sensing of CAUTIs, and the mechanism by which the fluorescent signal propagates from the sensors within the drainage bag were important considerations when formulating the prototype sensors. Above the self-quenching concentration when encapsulated within the hydrogel matrix, the CF is quenched owing to the formation of non-fluorescent dimers. Upon release of the dye into the urine diluent, the concentration is sufficiently lowered such that the self-quenching ceases and the fluorescence activated (FIG. 5A).

Suitability of CF dye for this application is further illustrated by the dependence of fluorescence intensity on pH (FIG. 5B). The drastic increase in fluorescence emission between pH 6-8 may be exploited such that the strongest photoluminescent signal is achieved in response to elevated urinary pH following infection by P. mirabilis. Maximum fluorescence output is observed at pH 7, corresponding to the onset of P. mirabilis infection.

### In Vitro Evaluation of Prototype Sensors: Bacterial Cultures

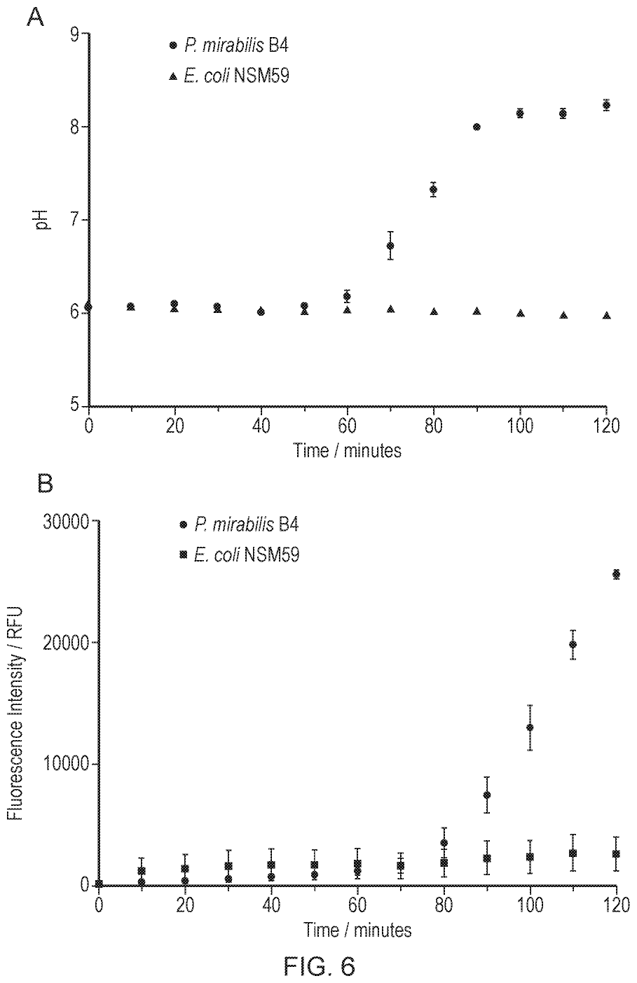

Initial assessment of the CF lozenge sensors in artificial urine supernatants of P. mirabilis and the urease-negative uropathogen E. coli confirmed dye release in response to elevated urinary pH (data not shown). Further analysis within live bacterial cultures exhibited selective release of CF in response to the onset of infection by P. mirabilis (FIG. 6).

Evaluation of sensor performance over a 2 hour period, within which the urinary pH was raised (FIG. 6A) from pH 6.1 (corresponding to an infection of 5.3×10⁶ CFU/ml) to 8.55 (corresponding to an infection of 2.3×10⁷ CFU/ml (data not shown)) was found to correspond to a rapid and observable increase in fluorescence response as a function of CF release from the hydrogel matrix (FIG. 6B). No significant fluorescence output was observed within the control culture of E. coli, owing to its inability to express urease, and hence, cause catheter blockage.

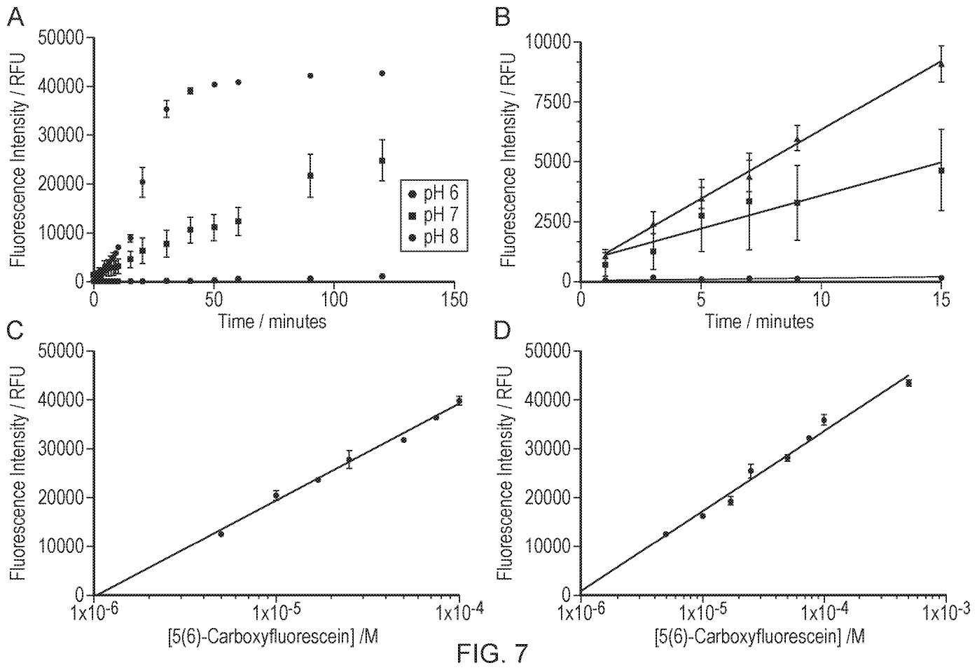

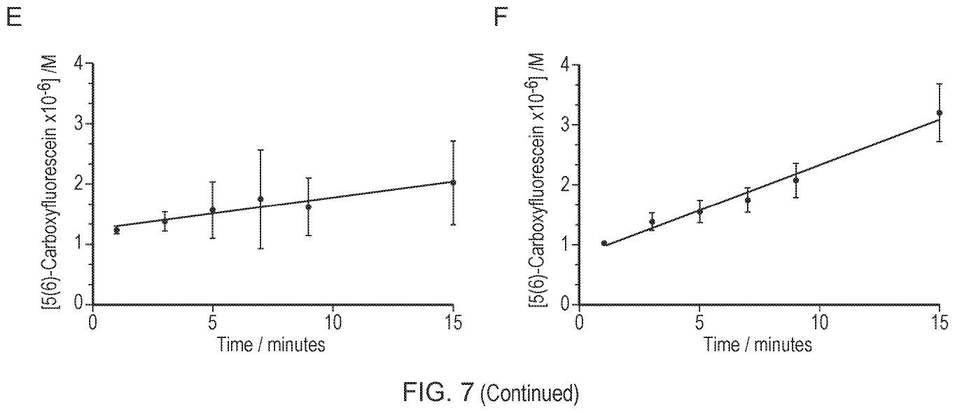

Investigation into the kinetics of initial CF-release from lozenge sensors (FIG. 7) revealed that the rate of release within the first 15 minutes of exposure to buffer at pH 7 was 5.3×10⁻⁸ mol dm⁻³ min⁻¹ at pH 7 (rising to 1.5×10⁻⁷ mol dm⁻³ min⁻¹ at pH 8) with a final CF concentration in urine of approximately 3.5 mM after 120 minutes. Hence, despite only approximately 14% of the encapsulated dye being released from the PVA reservoir, the resultant luminescence lies within the most fluorescent concentration region of the fluorescent chromophore (FIG. 1A). In effect, the conservation of the majority of the dye within the hydrogel sensor may help to retain dye signal strength over a longer period of time.

### Evaluation of Sensor Performance: Artificial Urine Supernatant

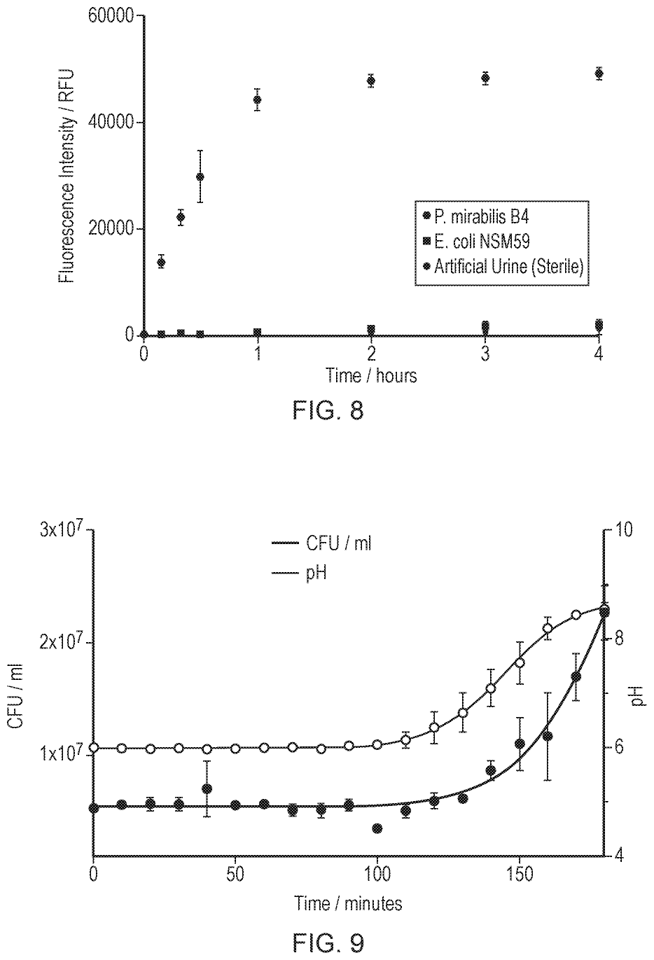

The ability of the sensors to release their fluorescent cargo in response to elevated artificial urine pH was evaluated using supernatant solutions of P. mirabilis B4 (average pH=8.98) and E. coli NSM59 (average pH=6.10). Sensor stability in sterile artificial urine was also evaluated, to ensure that no fluorescence output was obtained as a result of interactions between the Eudragit S100 ‘shell’, and the high ionic-strength artificial urine. Measured fluorescence intensity of artificial urine supernatants is shown in FIG. 8.

Since E. coli species are unable to express urease, the pH of the supernatant remained equivalent to that of the uninoculated artificial urine. Visual dye release was only observed for the urease-positive P. mirabilis supernatant, where the average urinary pH surpassed that of the Eudragit S100 swelling threshold.

### Evaluation of Sensor Performance: Artificial Urine Subcultures

### Correlation of Bacterial Bioburden with Urinary pH

The point at which sensor ‘switch on’ was achieved was evaluated using 1:1000 subcultures of P. mirabilis in artificial urine media. Urinary pH elevation to pH 8 was achieved in 150 minutes of incubation, owing to the expression of urease and subsequent urea hydrolysis within the media. The threshold pH at which the Eudragit ‘trigger’ layer undergoes swelling as a consequence of repulsion (pH 7) was achieved at approximately 135 minutes. This corresponded to the transition period of bacterial replication from lag phase to exponential growth phase (FIG. 9). Approximate bacterial concentration at this point was 7.45×10⁶ CFU/mL.

Since a substantial increase in urease production is associated with swarmer cell differentiation and expression of virulence factors, it is likely that P. mirabilis undergoes a coordinated virulence ‘switch on’ upon entering the exponential phase of growth. Indeed, previous investigation into the effects of the known anti-swarming agent p-nitrophenylglycerol (PNPG) revealed that the expression of virulence factors such as protease, urease haemolysin and flagellin were inhibited in addition to the transformation to hyperflaggerlate swarmer cell structure, upon incubation with PNPG.

### In Vitro Evaluation of Prototype Sensors: Laboratory Model of Catheterised Bladder

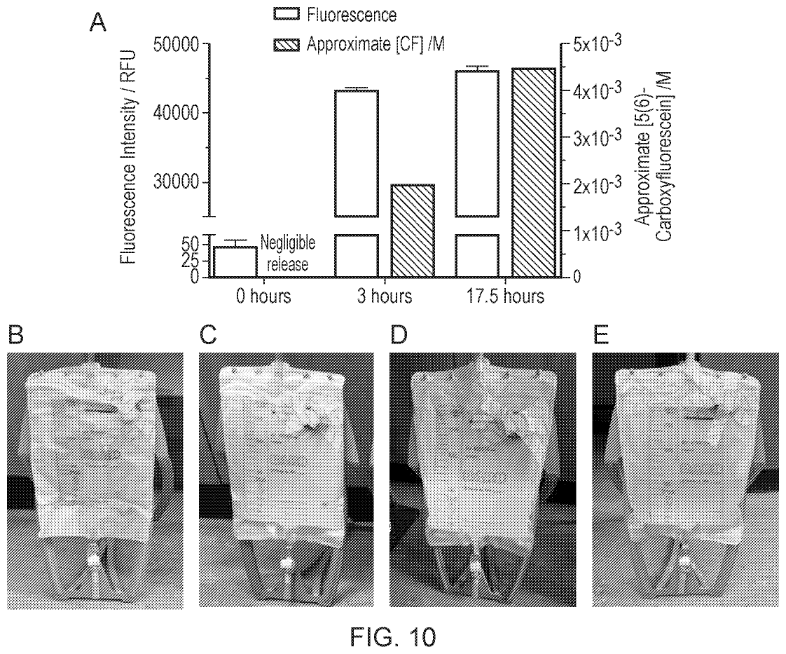

The performance of the dual-layered lozenge sensors (FIG. 1) to signal the onset of catheter blockage was tested using the in vitro bladder model system (originally described by Stickler et al., 1999), which replicates the full closed drainage system, and represents the in vivo catheterised urinary tract. Sensor ‘switch on’ was defined as the point at which a visual colour change was observed in residual urine within the drainage bag. Since the application of this technology requires the signal to be easily observable without the use of specialist equipment, the point of sensor switch on was subjectively judged by eye under ambient lighting. Observation of visual dye release prompted quantitative analysis of urinary fluorescence (FIG. 10A), such that the concentration of CF in the diluent could be estimated. The response of lozenge sensors to E. coli was also assessed to determine the selectivity of the sensors towards urease-negative species.

Dye release and consequent visual urinary colour change within the drainage bags (data not shown) was observed after 3 hours (urine pH 7.2), corresponding to approximately 2 mM CF released (doubling by the point of blockage to ˜4 mM) (FIG. 10A). Average blockage time of P. mirabilis infected models occurred at 17.5 hours after the flow of urine to bladders was initiated (urine pH 8.6). Hence, the CF lozenge sensors achieved an average early warning of catheter blockage of 14.5 hours, with a range of 12.5-18 hours. The appearance and progression of a clear and unambiguous colour change (FIGS. 10B-D) within the drainage bag was comparable with results seen previously (Malic et al., 2012; Milo et al., 2016), and was deemed to be sufficient to allow intervention before the manifestation of serious clinical complications. Control bladders inoculated with E. coli exhibited negligible fluorescence emission throughout the duration (23 hours) of the experiment (FIG. 10E).

The rapid catheter blockage (17.5 hours) observed using the in vitro model of the catheterised bladder is achieved via inoculation with a large bacterial bioburden (10⁸ CFU/ml P. mirabilis B4) at the experimental start. This endeavours to replicate conditions within the catheterised urinary tract of patients who frequently undergo recurrent catheter blockage, whereby the blocked catheter is removed and quickly replaced directly into urine cultures of P. mirabilis at alkaline pH containing aggregates of microcrystalline material (Stickler and Feneley, 2010). The problem of blockage recurrence affects up to 50% of patients undergoing long-term indwelling catheterisation, and sufferers may experience occlusion as soon as 2 days post-catheter reinsertion. Since the sensors tested under these ‘worst case scenario’ conditions signalled catheter blockage sufficiently early (14.5 hours) to permit clinical intervention, they provide a simple and convenient potential management system for recurring urinary catheter blockage, a problem for which there is no current control method.

The sensor design described in this work provides advantages over sensing approaches described previously, due to the specific dissolution pH threshold of the EUDRAGIT® S100 trigger layer. Since the median healthy urinary pH is 6.2 with a mean range of 5.5-7.0, and the average voided urinary pH of patients with consistently blocking catheters is 7.85, the rapid and reproducible dissolution of EUDRAGIT® S100 above pH 7 allows for a less ambiguous result with potentially fewer false positives when compared to the previously described bromothymol blue-based sensor (Malic et al., 2012), which responded to increasing urinary pH over a broad range (pH 6-8).

The design constraints of the current Foley catheter system were also important considerations in the design of the described sensor. Since the current Foley catheter design has remained unchanged for decades, any technological advances in the control of CAUTI requiring the fabrication of additional components or redesign of the catheter or closed drainage system may well be rejected by patients and carers alike, owing to the introduction of additional cost or complexity. Indeed, some of these issues were highlighted in the pilot scale clinical trials of the bromothymol blue sensor (Long et al., 2014), where the infection indicator was inserted as a connector in the junction between the catheter and drainage bag. Some participants reported leakage around the sensor, or expressed concern over the additional length (Long et al., 2014). In contrast, the work described here fits well with existing manufacturing processes and all available drainage bag designs, although the insertion of the sensor into the bag would need to occur at the point of manufacture, such that the sterility of the closed drainage system is not compromised at the point-of-care. Perhaps the most important advantage of the bag-based system employed here is the potential to incorporate antimicrobial coatings to the catheter tip, for example those containing bacteriophage (Milo et al., 2017) or metal oxide nanoparticles (Shalom et al., 2017) to create a theranostic system whereby the onset of blockage is delayed by the therapeutic coating (thus extending catheter lifetime), and the subsequent elution/failure of the antimicrobial is signalled by the diagnostic sensor visible within the drainage bag.

### Conclusion

In summary, this work describes the development of a simple, drainage bag-based sensor for the early warning of catheter blockage following infection by P. mirablis. Since many patients undergoing long-term indwelling catheterisation are in community care, the collection and analysis of urine samples can be problematic. The system described in this work provides a convenient alternative, allowing the patient or carer to be informed of imminent blockage such that appropriate intervention may be facilitated. Evaluation of coating performance in a laboratory model of the catheterised bladder showed that lozenge sensors permitted 14.5 hours warning of catheter blockage under conditions of established infection. Overall, sensor performance was comparable to other reported sensor systems, although the implementation of this approach provides significant advantages both in terms of response location and manufacturing considerations.

### REFERENCES

Long, A., Edwards, J., Thompson, R., Lewis, D. A., Timoney, A. G., 2014. A clinical evaluation of a sensor to detect blockage due to crystalline biofilm formation on indwelling urinary catheters. BJU International 114, 278-285. doi:10.1111/bju.12577

Malic, S., Waters, M. G. J., Basil, L., Stickler, D. J., Williams, D. W., 2012. Development of an ‘early warning’ sensor for encrustation of urinary catheters following Proteus infection. Journal of Biomedical Materials Research—Part B Applied Biomaterials 100 B, 133-137.

Milo, S., Thet, N. T., Liu, D., Nzakizwanayo, J., Jones, B. V., Jenkins, A. T. A., 2016. An in-situ infection detection sensor coating for urinary catheters. Biosensors and Bioelectronics 81, 166-172. doi: 10.1016/j.bios.2016.02.059

Milo, S., Hathaway, H., Nzakizwanayo, J., Alves, D. R., Esteban, P. P., Jones, B. V., Jenkins, A. T. A., 2017. Prevention of encrustation and blockage of urinary catheters by Proteus mirabilis via pH-triggered release of bacteriophage. Journal of Materials Chemistry B 5, 5403-5411. doi:10.1039/C7TB01302G

Shalom, Y., Perelshtein, I., Perkas, N., Gedanken, A., Banin, E., 2017. Catheters coated with Zn-doped CuO nanoparticles delay the onset of catheter-associated urianry tract infections. Nano Research 10, 520-533. doi:10.1007/s12274-016-1310-8

Stickler, D., Morris, N., Winters, C., 1999. Simple Physical Model to Study Formation and Physiology of Biofilms on Urethral Catheters. Methods in Enzymology 310, 498-501. doi:10.1016/50076-6879(99)10037-5

Stickler, D. J., Jones, S. M., Adusei, G. O., Waters, M. G., 2006a. A sensor to detect the early stages in the development of crystalline Proteus mirabilis biofilm on indwelling bladder catheters. Journal of Clinical Microbiology 44, 1540-1542. doi:10.1128/JCM.44.4.1540-1542.2006

Stickler, D. J., Jones, S. M., Adusei, G. O., Waters, M. G., Cloete, J., Mathur, S., Feneley, R. C. L., 2006b. A clinical assessment of the performance of a sensor to detect crystalline biofilm formation on indwelling bladder catheters. BJU International 98, 1244-1249. doi:10.1111/j.1464-410X.2006.06562.x

Stickler, D. J., Feneley, R. C. L., 2010. The encrustation and blockage of long-term indwelling bladder catheters: a way forward in prevention and control. Spinal Cord 48, 784-790. doi:10.1038/sc.2010.32

1. A sensor for locating within a urinary drainage bag, the sensor comprising an agent held by a pH-sensitive release material;
wherein the agent is a diagnostic marker and wherein the pH-sensitive release material is configured to release the agent when the pH-sensitive release material is exposed a pH above 7.

2. The sensor according to claim 1, wherein the sensor comprises an inner layer containing the diagnostic marker and an outer layer comprising the pH-sensitive release material.

3. The sensor according to claim 2, wherein the inner layer comprises a hydrogel.

4. The sensor according to claim 3, wherein the hydrogel comprises one or more of poly(vinyl alcohol), agarose, Hypromellose (HPMC), gelatine or sodium polyacrylate.

5. The sensor according to claim 2, wherein the inner layer comprises the diagnostic marker encapsulated in a gelatine or Hypromellose (HPMC) capsule.

6. The sensor according to claim 1, wherein the pH-sensitive release material is a pH-sensitive polymer comprising carboxyl and ester groups.

7. The sensor according to claim 6, wherein the pH-sensitive polymer comprises carboxyl and ester groups at a ratio of 1:2.

8. The sensor according to claim 6, wherein the pH-sensitive polymer comprises poly(methyl methacrylate-co-methacrylic acid).

9. The sensor according to claim 1, wherein the diagnostic marker is a dye.

10. The sensor according to claim 9, wherein the dye selected from one or more of fluorescein, carboxyfluorescein or sulforhodamine B.

11. A urinary drainage bag comprising a sensor according to claim 1.

12. A urinary catheter comprising a drainage bag according to claim 11.

13. A method of preventing urinary catheter blockage due to bacterial colonisation, the method comprising the steps of:
fitting a urinary catheter according to claim 12 to a patient; and
monitoring release of the diagnostic marker,
wherein release of the diagnostic marker indicates bacterial colonisation.

14. The method of claim 13, wherein the diagnostic marker is released about 12 to about 18 hours prior to catheter blockage.

15. The method of claim 13, wherein release of the diagnostic marker provides a visible color change of residual urine in the drainage bag.

16. The method of claim 13, wherein the bacterial colonisation is due to a urinary tract infection.

17. The method of claim 16, wherein the urinary tract infection is a urease producing bacterial infection.

18. The method of claim 17, wherein the urease producing bacteria is Proteus mirabilis, Providencia rettgeri, Morganella morganii, Pseudomonas aeruginosa, Proteus vulgaris or a combination thereof.

19. The method of claim 13, the method further comprising removing the catheter from the patient after release of the diagnostic marker.

20. The method of claim 19, wherein the catheter is removed from the patient within about 18 hours of release of the diagnostic marker.

21. A method for manufacturing a sensor according to claim 3, the method comprising:
combining a solution of a diagnostic marker and a hydrogel polymer and heating the solution to form a hydrogel containing the diagnostic marker,
cooling the hydrogel containing the diagnostic marker to about −20° C. to promote cryogenic gelation,
thawing the hydrogel containing the diagnostic marker at room temperature, and
coating the thawed hydrogel containing the diagnostic marker with a pH-sensitive release material.

22. The method of claim 21, wherein the thawed hydrogel containing the diagnostic marker is coated by dip coating or by filling a capsule with the thawed hydrogel containing the diagnostic marker and tablet coating the filled capsule.
